(19) United States
(12) Patent Application Publication
(10) Pub. No.: US 2015/0138538 A1
(43) Pub. Date: May 21, 2015

(54) COMPONENT ANALYZING APPARATUS

(71) Applicant: Seiko Epson Corporation, Tokyo (JP)

(72) Inventor: Kazunori SAKURAI, Chino (JP)

(21) Appl. No.: 14/540,481

(22) Filed: Nov. 13, 2014

(30) Foreign Application Priority Data
Nov. 20, 2013 (JP) 2013-239661

(51) Int. Cl. G01N 21/25; G01N 33/02; G01G 19/00; G01J 3/28

(52) U.S. Cl. CPC G01N 21/255; G01J 3/2823; G01N 33/02; G01G 19/00; G01N 2201/062; G01N 2201/06113
USPC 356/72

(57) ABSTRACT

A component analyzing apparatus includes a base unit having a mounting part on which a measuring object is mounted and a mass measurement part that measures a mass of the measuring object, a spectroscopic camera that acquires a spectral image of the measuring object, a support unit provided on the base unit and supporting the spectroscopic camera in a location such that an imaging direction in the spectroscopic camera is a direction toward the mounting part and a distance between the mounting part and the spectroscopic camera is a predetermined distance, and a control part that analyzes components of the measuring object based on the spectral image and the mass measured by the mass measurement part.

### BACKGROUND

1. Technical Field

The present invention relates to a component analyzing apparatus etc.

2. Related Art

Recently, calorie measuring apparatuses for measuring calories of measuring objects have been known (for example, see Patent Document 1 (JP-A-2009-098015)).

An apparatus disclosed in Patent Document 1 measures calories of a food as a measuring object mounted on a table provided in a chamber using calorie measuring means. In the apparatus, the chamber has an infrared light blocking film that blocks near-infrared light and an electromagnetic wave blocking member that blocks electromagnetic wave, and external infrared light and electromagnetic wave are blocked. Further, the apparatus includes a mass measuring device for measuring the mass of the measuring object mounted on the table. The calorie measuring means applies light in a near-infrared range to the measuring object within the chamber and receives the reflection light or transmission light with a light receiving unit. Then, the calorie measuring means calculates calories of the object to be measured based on the amount of received light in the light receiving unit and the measured mass.

In the above described calorie measuring apparatus in Patent Document 1, it is necessary to house the measuring object in the chamber that prevents intrusion of outside light and electromagnetic wave, and there is a problem of upsizing of the apparatus.

On the other hand, small calorie measuring apparatuses have been known (for example, see Patent Document 2 (JP-A-2012-112855)).

A system disclosed in Patent Document 2 includes a portable scale and a digital camera that can be connected to the portable scale using a cable. In the system, a measuring object is mounted on the portable scale and its image is taken by the digital camera. Then, the ingested weight and ingested calories of a meal of the user are estimated using the weight of the measuring object measured by the portable scale and the image taken by the digital camera.

In the above described system disclosed in Patent Document 2, it is necessary to hold the digital camera with hands for imaging when the image of the measuring object on the portable scale is taken using the digital camera. Accordingly, the distance from the digital camera to the portable scale changes at each time of imaging, and there is a problem that it is difficult to accurately measure the size of the measuring object. For the problem, in Patent Document 2, the size is detected using a size indicator as a measurement reference. However, it is necessary to set the size indicator at each time of measurement and there is a problem that the operation is troublesome. Further, in the portable calorie measuring apparatus, carrying the size indicator at every time is troublesome, and there is a problem that the measurement accuracy is lower when the size indictor is not carried.

### SUMMARY

An advantage of some aspects of the invention is to provide a small component analyzing apparatus that can perform high-accuracy component analysis.

A component analyzing apparatus according to an aspect of the invention includes a base unit having a mounting part on which a measuring object is mounted and a mass measurement part that measures a mass of the measuring object, a spectroscopic imaging unit that acquires a spectral image of the measuring object, a support unit provided in the base unit and supporting the spectroscopic imaging unit in a location in which an imaging direction in the spectroscopic imaging unit is a direction toward the mounting part and a distance between the mounting part and the spectroscopic imaging unit is a predetermined distance, and a component analysis part that analyzes components of the measuring object based on the spectral image and the mass measured by the mass measurement part.

In the aspect of the invention, the mass measurement part is provided in the mounting part of the base unit, and thereby, the correct mass of the measuring object may be measured by mounting the measuring object on the mounting part. Further, the support unit is provided on the base unit, and the spectroscopic imaging unit is supported by the support unit so that the imaging direction may be directed toward the mounting part. In the configuration, compared to the case where the measuring object is imaged with a spectroscopic camera in hand, for example, there is less blur of the spectral image due to camera shake or the like. Further, the locations of the spectroscopic imaging unit and the mounting part are kept at the predetermined distance, and thereby, the correct size of the measuring object may be calculated from the spectral image without using a size indicator or the like, for example. Therefore, the component analysis part may analyze the components of the measuring object with high accuracy based on the spectral image, the mass, and the size of the measuring object.

Further, the spectroscopic imaging unit is supported by the support unit with respect to the base unit, and, compared to an apparatus in which the mounting part is provided within a casing having a chamber, for example, simplification of the component may be realized.

In the component analyzing apparatus according to the aspect of the invention, it is preferable that the support unit has a distance change unit that changes the distance between the mounting part and the spectroscopic imaging unit, and the component analyzing apparatus includes a distance measurement unit that measures the distance between the mounting part and the spectroscopic imaging unit.

In the aspect of the invention with this configuration, the distance between the mounting part and the spectroscopic imaging unit may be adjusted, and thereby, when the measuring object lies out of the spectral image, for example, the adjustment of extending the distance between the mounting part and the spectroscopic imaging unit such that the measuring object may be within the spectral image or the like may be performed. Further, even when the distance is adjusted in the above described manner, the correct size of the measuring object may be measured using the distance measured by the distance measurement unit and the spectral image.

In the component analyzing apparatus according to the aspect of the invention, it is preferable that the distance change unit includes a position change part that changes a position of the support unit with respect to the base unit.

In the aspect of the invention with this configuration, the position change part that changes the position of the support unit is provided, and thereby, the imaging angle with respect to the measuring object by the spectroscopic imaging unit may be adjusted by changing the position of the support unit.

In the component analyzing apparatus according to the aspect of the invention, it is preferable that the distance change unit includes a position detection unit that detects the position of the support unit with respect to the base unit, and the distance measurement unit calculates the distance between the spectroscopic imaging unit and the mounting part based on the detected position.

In the aspect of the invention with this configuration, the position of the support unit with respect to the base unit (e.g., the rotation angle of the support unit with respect to the base unit) is detected and the distance between the spectroscopic imaging unit and the mounting part is calculated based on the position. In the support unit, if the length from the support location of the spectroscopic imaging unit and the connecting location between the support unit and the base unit is known, the distance between the spectroscopic imaging unit and the mounting part may be easily calculated based on the detected position of the support unit.

In the component analyzing apparatus according to the aspect of the invention, it is preferable that a direction detection unit that detects the imaging direction of the spectroscopic imaging unit is provided, and the distance measurement unit calculates the distance between the spectroscopic imaging unit and the mounting part based on the detected imaging direction.

In the aspect of the invention with this configuration, the distance between the spectroscopic imaging unit and the mounting part is calculated based on the imaging direction detected by the direction detection unit. That is, if the length from the support location of the spectroscopic imaging unit and the connecting location between the support unit and the base unit is known, the distance between the spectroscopic imaging unit and the mounting part may be easily calculated based on the imaging direction. Further, the more correct distance from the spectroscopic imaging unit to the mounting part may be calculated based on the position of the support unit detected by the above described position detection unit and the imaging direction.

In the component analyzing apparatus according to the aspect of the invention, it is preferable that the support unit includes a plurality of partial support parts rotatably coupled to each other.

In the aspect of the invention with this configuration, the support unit includes the plurality of partial support parts such as arms, for example. Accordingly, the angles of these partial support parts are adjusted, and thereby, the location of the spectroscopic imaging unit may be finely controlled.

In the component analyzing apparatus according to the aspect of the invention, it is preferable that the support unit is rotatably provided with respect to the base unit, and the base unit includes a housing part in which the support unit can be folded and housed when the support unit is rotated toward the base unit side.

In the aspect of the invention with this configuration, the support unit is housed in the housing part, and thereby, the component analyzing apparatus may be made even smaller and improvement in portability may be realized.

In the component analyzing apparatus according to the aspect of the invention, it is preferable that a light source part provided in a part of the support unit and applying light to the measuring object is provided.

In the aspect of the invention with this configuration, the light source part is in a part of the support unit, and the application direction of the light from the light source part may be changed by changing the position of the support unit. When the spectral image is taken by the spectroscopic imaging unit, if a part in which the light from the light source part is regularly reflected exists, it may be impossible to acquire the correct optical spectrum in the part and perform a component analysis with high accuracy. On the other hand, in the aspect of the invention with the configuration described above, the angle of the support unit is changed and the application direction is changed in the above described manner, and thereby, the regular reflection part may be changed. That is, the application direction from the light source part may be appropriately corrected so that the regular reflection part may not be produced.

Further, the correct optical spectrum may be acquired by using two spectral images having different regular reflection parts and replacing the optical spectrum in the regular reflection part of one spectral image by the optical spectrum in the corresponding location of the other spectral image.

In the component analyzing apparatus according to the aspect of the invention, it is preferable that the spectroscopic imaging unit is detachably provided from the support unit.

In the aspect of the invention with this configuration, the spectroscopic imaging unit is detachable from the support unit, and thereby, maintenance of the spectroscopic imaging unit may be preferably performed.

In the component analyzing apparatus according to the aspect of the invention, it is preferable that the component analysis part is provided in the base unit or the support unit, and has a first communication part that makes wireless communication with the spectroscopic imaging unit, and the spectroscopic imaging unit has a second communication part that makes wireless communication with the first communication part.

In the aspect of the invention with this configuration, even when the spectroscopic imaging unit is separated from the support unit as described above, the taken image imaged by the spectroscopic imaging unit may be transmitted to the component analysis part.

In the component analyzing apparatus according to the aspect of the invention, it is preferable that the mounting part has a reference part having reference reflectance.

In the aspect of the invention with this configuration, calibration is performed using the reference part, and thereby, even when outside light exists, for example, the optical spectrum of the measuring object may be accurately acquired and the component analysis with high accuracy may be performed. Further, it is not necessary to prepare another reference object for calibration, and simplification of the configuration may be realized.

Specifically, it is preferable that a plurality of reference parts having different reflectances for the respective wavelengths (from near-infrared to infrared wavelength ranges) as measuring objects including a first reference part having the higher reflectances for the respective wavelengths and a second reference part having the lower reflectances for the respective wavelengths are provided.

In the component analyzing apparatus according to the aspect of the invention, it is preferable that the spectroscopic imaging unit includes a tunable Fabry-Perot etalon that light from the measuring object enters, selects light having a predetermined wavelength from the incident light, and can change the predetermined wavelength, and a light receiving element that receives the light output from the Fabry-Perot etalon.

In the aspect of the invention with this configuration, the spectroscopic imaging unit selects the light having the predetermined wavelength using the tunable Fabry-Perot etalon and receives the output light using the light receiving element. The Fabry-Perot etalon may be simply formed by providing a pair of reflection films to be opposed, and the spectral wavelength may be easily changed by changing the gap dimension between the reflection films. Therefore, the tunable Fabry-Perot etalon is used, and thereby, compared to the case where the large spectroscopic unit like an AOTF (acousto-optic tunable filter) or an LCTF (liquid crystal tunable filter) is used, for example, the spectroscopic imaging unit and the component analyzing apparatus may be made smaller.

In the component analyzing apparatus according to the aspect of the invention, it is preferable that a memory unit in which correlation data between a feature quantity extracted from an absorption spectrum of a component to be analyzed and a component content rate of the component to be analyzed is stored, and, when a plurality of the measuring objects are mounted on the mounting part, the component analysis part calculates the content rates of the component to be analyzed contained in the respective measuring objects calculated based on amounts of light of the respective pixels of the spectral image and the correlation data, calculates estimated volumes of the respective measuring objects calculated based on the distance between the mounting part and the spectroscopic imaging unit, estimated weights of the component to be analyzed contained in the measuring objects based on the content rates of the component to be analyzed contained in the respective measuring objects, and estimated weights of the respective measuring objects, and corrects the estimated weights of the component to be analyzed contained in the respective measuring objects based on a sum of the estimated weights of the respective measuring objects and a ratio to the mass measured by the mass measurement part.

In the aspect of the invention with this configuration, even when the plurality of the measuring objects are mounted on the mounting part, the contents of the respective components contained in the respective measuring objects may be corrected based on the correct masses as described above. That is, when the plurality of the measuring objects are mounted on the mounting part, only the sum of the masses of these measuring objects is measured by the mass measurement part, and the masses of the respective measuring objects are unknown. On the other hand, in the aspect of the invention with the configuration described above, since the estimated masses of the respective components, and the respective measuring objects may be obtained based on the estimated volumes estimated based on the distance, the appropriate correction may be performed based on the sum of these estimated masses and the measured masses, and thereby, it is unnecessary to individually perform the component analyses for the respective measuring objects.

### BRIEF DESCRIPTION OF THE DRAWINGS

The invention will be described with reference to the accompanying drawings, wherein like numbers reference like elements.

FIG. 1 is a perspective view showing a schematic configuration of a component analyzing apparatus of the first embodiment.

FIG. 2 is a block diagram showing a system configuration in the component analyzing apparatus of the first embodiment.

FIG. 3 shows a schematic configuration of abase unit and a support unit of the first embodiment.

FIG. 4 shows a schematic configuration of a spectroscopic camera of the first embodiment.

FIG. 5 is a plan view showing a schematic configuration of a tunable interference filter of the first embodiment.

FIG. 6 is a sectional view along line A-A in FIG. 5.

FIG. 7 is a flowchart showing a component analysis method of the component analyzing apparatus of the first embodiment.

FIG. 8 is a flowchart showing a component analysis method of a component analyzing apparatus of the second embodiment.

FIG. 9 is a schematic diagram showing a position change of a support unit of the second embodiment.

FIGS. 10A to 10C show examples of a first spectral image, a second spectral image in corrected size, and a corrected spectral image in the second embodiment.

FIG. 11 is a perspective view showing a schematic configuration of a component analyzing apparatus of the third embodiment.

FIG. 12 schematically shows a support unit of the third embodiment.

FIG. 13 is a schematic diagram showing an example of a component analyzing apparatus of a modified example in the invention.

FIGS. 14A and 14B are perspective views showing another example of a component analyzing apparatus of a modified example in the invention.

### DESCRIPTION OF EXEMPLARY EMBODIMENTS

### First Embodiment

As below, a component analyzing apparatus according to the first embodiment of the invention will be explained with reference to the drawings.

### Schematic Configuration of Component Analyzing Apparatus

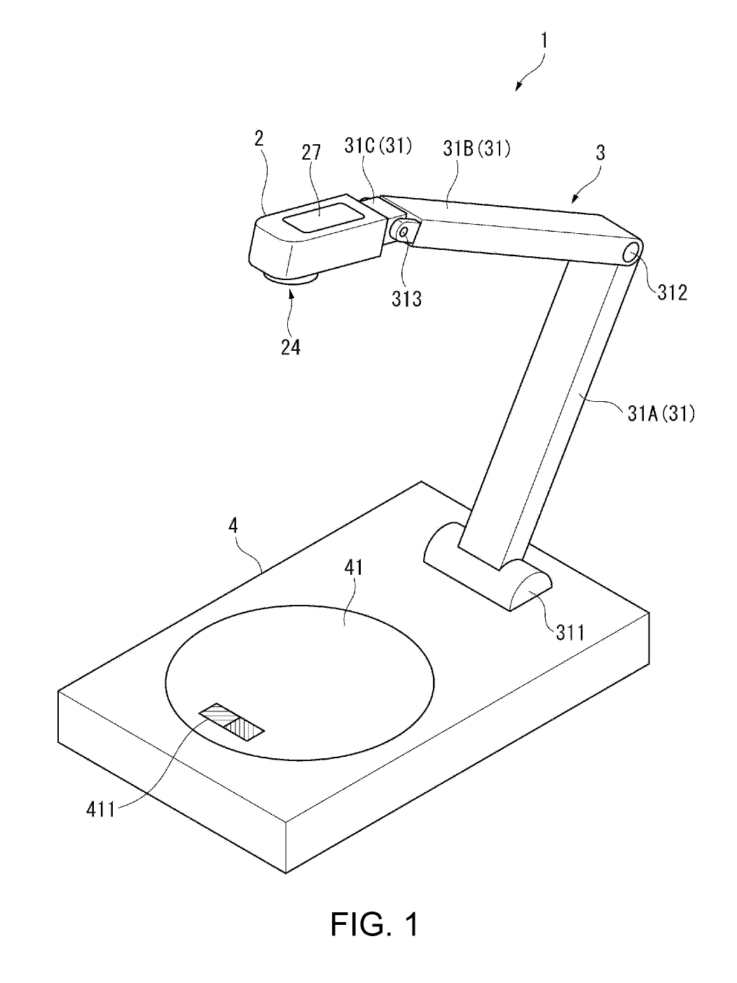

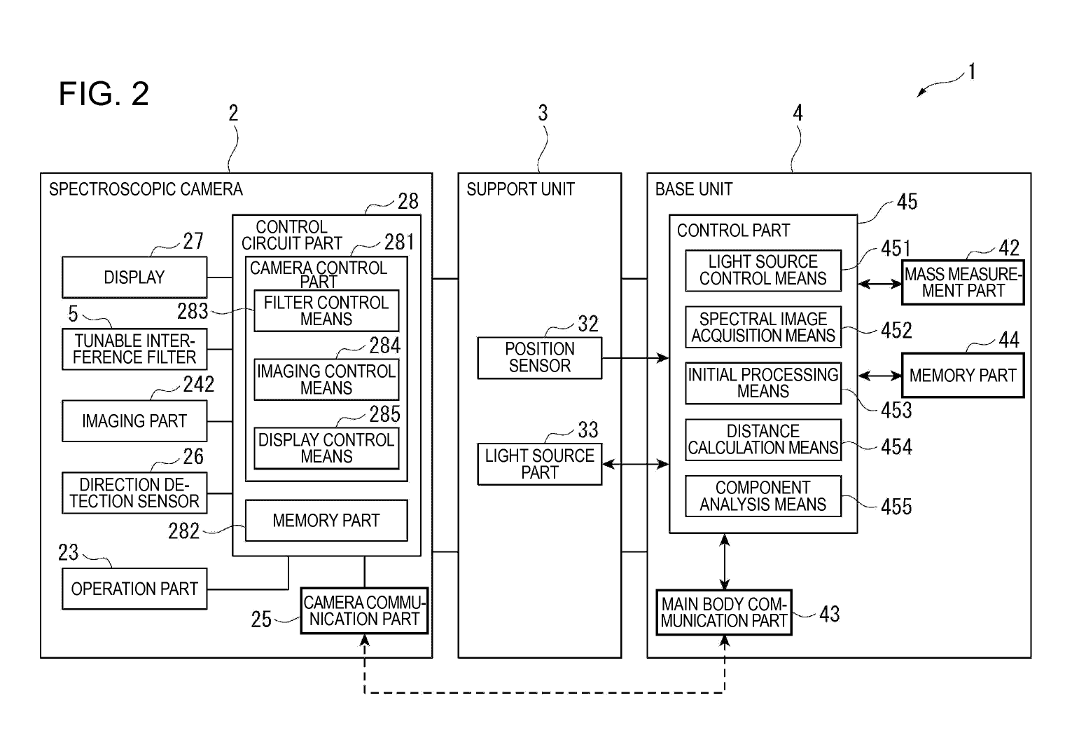

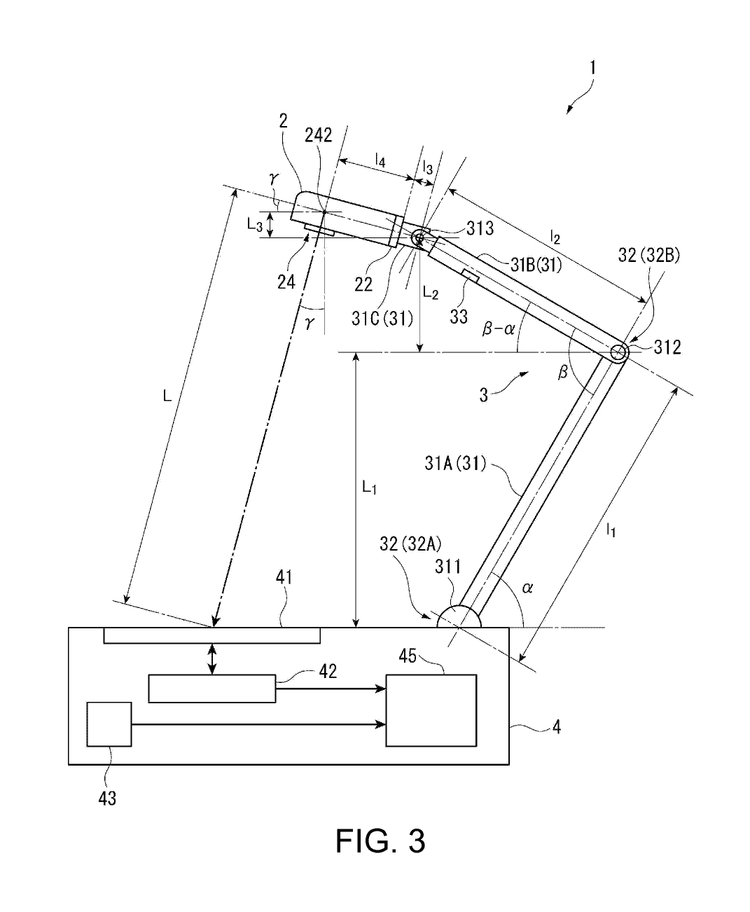

FIG. 1 is a perspective view showing a schematic configuration of a component analyzing apparatus 1 of the first embodiment. FIG. 2 is a block diagram showing a system configuration in the component analyzing apparatus 1 of the embodiment. FIG. 3 shows a schematic configuration of a base unit 4 and a support unit 3.

As shown in FIG. 1, the component analyzing apparatus 1 includes a spectroscopic camera 2, the support unit 3 to which the spectroscopic camera 2 is detachably attached, and the base unit 4 to which the support unit 3 is connected.

### Configuration of Spectroscopic Camera

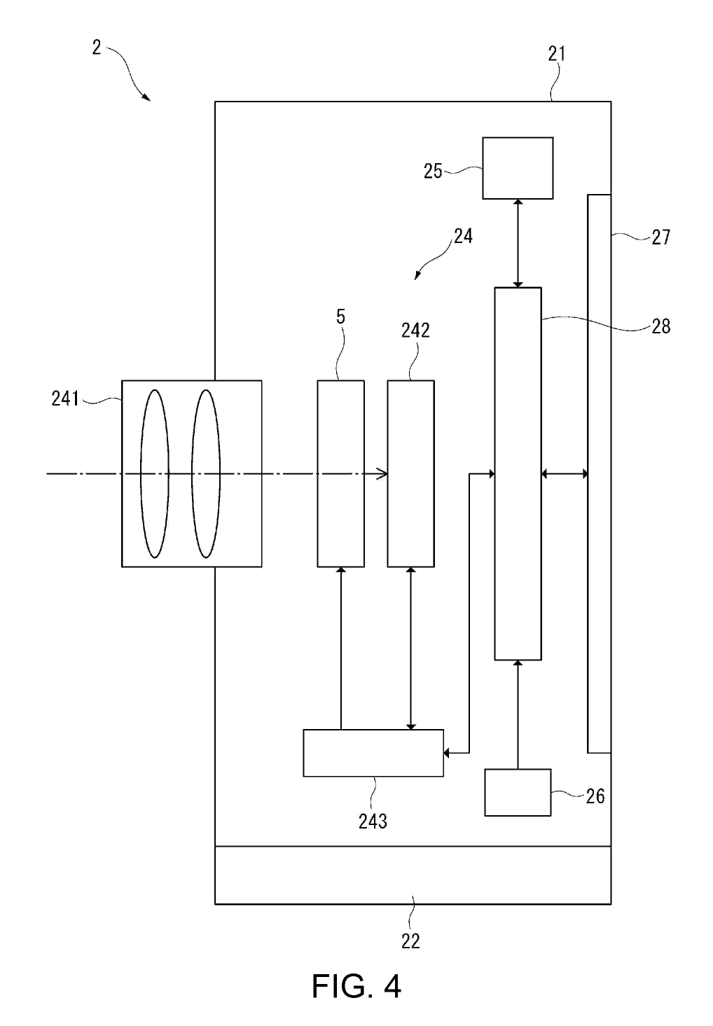

FIG. 4 shows a schematic configuration of the spectroscopic camera 2 in the embodiment.

As shown in FIG. 4, the spectroscopic camera 2 forms a spectroscopic imaging unit according to the invention, and includes a casing 21, an attachment part 22, an operation part 23 (see FIG. 2), an imaging module 24, a camera communication part 25 as a second communication part according to the invention, a direction detection sensor 26 (direction detection unit), a display 27, and a control circuit part 28.

As the attachment part 22, any configuration may be employed as long as the configuration can be detachably attached to the support unit 3, for example. The attachment part 22 includes a configuration in which an engagement hole is provided and an engagement pin provided on the support unit 3 is engaged, and thereby, the attachment part 22 is attached to the support unit 3, and a configuration in which a male screw portion provided in the support unit 3 is screwed in a threaded hole provided in the casing 21.

### Configuration of Operation Part

The operation part 23 is provided in a part of the casing 21.

The operation part 23 includes a shutter button provided in the casing 21, a touch panel provided on the display 27, etc. Further, when an input operation is performed by a user, the operation part 23 outputs an operation signal in response to the input operation to the control circuit part 28.

### Configuration of Imaging Module

The casing 21 includes an imaging window and the imaging module 24 is provided to face the imaging window. The imaging module 24 includes a light incident part 241, a tunable interference filter 5 (tunable Fabry-Perot etalon), an imaging part 242 that receives incident light (light receiving element according to the invention), and a driver circuit 243 that controls the tunable interference filter 5 and the imaging part 242.

### Configuration of Light Incident Part

The light incident part 241 includes a plurality of lenses, for example. It is preferable that these lenses form a telecentric optical system, limit the viewing angle to a predetermined angle or less, and form an image of the measuring object within the viewing angle in the imaging part 242. The telecentric optical system is used, and thereby, the optical axis of the incident light may be aligned in a direction in parallel to the principal ray and the incident light may be allowed to perpendicularly enter a fixed reflection film 54 and a movable reflection film 55 of the tunable interference filter 5, which will be described later. Further, a diaphragm is provided in a focal position of the lenses, and the diaphragm diameter is controlled in response to the user operation or the like, for example, and thereby, the viewing angle may be controlled.

Further, it is preferable that a magnification/reduction optical system is additionally provided in the light incident part 241. The magnification/reduction optical system is provided and the lens intervals are adjusted in response to the user operation, for example, and thereby, the acquired images may be magnified and reduced.

### Configuration of Tunable Interference Filter

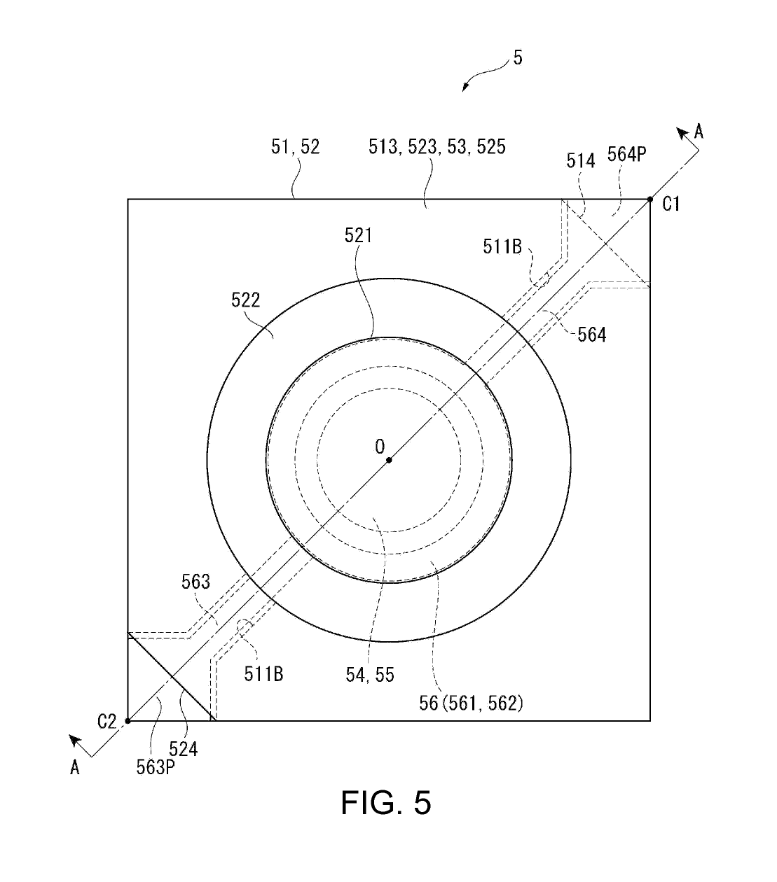

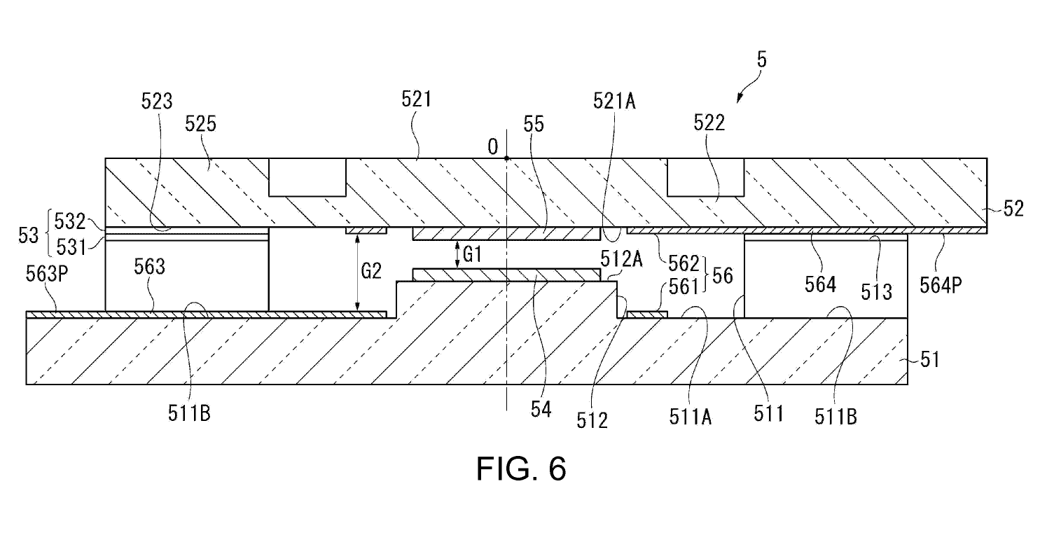

FIG. 5 is a plan view showing a schematic configuration of the tunable interference filter. FIG. 6 is a sectional view of the tunable interference filter along line A-A in FIG. 5.

The tunable interference filter 5 is a Fabry-Perot etalon and includes a pair of substrates (a fixed substrate 51 and a movable substrate 52). These substrates 51, 52 are respectively formed using various kinds of glass including soda glass, crystalline glass, quartz glass, lead glass, potassium glass, borosilicate glass, and alkali-free glass, or crystal, for example. Note that, in the embodiment, for acquisition of spectral images of the measuring object in the infrared range, the substrates 51, 52 may be formed using silicon or the like that can transmit light in the infrared range.

Further, these fixed substrate 51 and movable substrate 52 are joined by a joining film 53 including a plasma-polymerized film primarily consisting of siloxane, for example, and thereby, integrally formed.

Furthermore, the fixed reflection film 54 is provided on the fixed substrate 51 and the movable reflection film 55 is provided on the movable substrate 52. These fixed reflection film 54 and movable reflection film 55 are provided to be opposed via a gap between reflection films G1. In addition, an electrostatic actuator 56 used for adjustment (change) of the amount of gap of the gap between reflection films G1 is provided in the tunable interference filter 5. The electrostatic actuator 56 includes a fixed electrode 561 provided on the fixed substrate 51 and a movable electrode 562 provided on the movable substrate 52. These fixed electrode 561 and movable electrode 562 are opposed via a gap between electrodes G2. Here, these fixed electrode 561 and movable electrode 562 may be directly provided on the substrate surfaces of the fixed substrate 51 and the movable substrate 52, respectively, or may be provided via other film members.

Further, in the tunable interference filter 5 in the embodiment, in the plan view as seen from the substrate thickness direction of the fixed substrate 51 (movable substrate 52) (hereinafter, referred to as “filter plan view”) as shown in FIG. 5, the plane center point O of the fixed substrate 51 and the movable substrate 52 coincides with the center point of the fixed reflection film 54 and the movable reflection film 55 and coincides with the center point of a movable part 521, which will be described later.

### Configuration of Fixed Substrate

On the fixed substrate 51, an electrode placement groove 511 and a reflection film provision part 512 are formed by etching or the like, for example. Further, a cutout part 514 is formed at an apex C1 of the fixed substrate 51, and a movable electrode pad 564P to be described later is exposed when the tunable interference filter 5 is seen from the fixed substrate 51 side.

The electrode placement groove 511 is annularly formed around the plane center point O of the fixed substrate 51 in the filter plan view. The reflection film provision part 512 is formed to project toward the movable substrate 52 side from the center part of the electrode placement groove 511 in the plan view. The groove bottom surface of the electrode placement groove 511 serves as an electrode provision surface 511A on which the fixed electrode 561 is provided. Further, the projected end surface of the reflection film provision part 512 serves as a reflection film provision surface 512A.

Further, on the fixed substrate 51, an electrode extraction groove 511B extending from the electrode placement groove 511 toward the apex C1 and an apex C2 of the outer circumference edge of the fixed substrate 51 is provided.

The fixed electrode 561 is provided on the electrode provision surface 511A of the electrode placement groove 511. More specifically, the fixed electrode 561 is provided in a region opposed to the movable electrode 562 of the movable part 521, which will be described later, of the electrode provision surface 511A. Further, an insulating film for securing insulation between the fixed electrode 561 and the movable electrode 562 may be stacked on the fixed electrode 561.

Further, on the fixed substrate 51, a fixed extraction electrode 563 extending from the outer circumference edge of the fixed electrode 561 in the direction toward the apex C2 is provided. The extending end part (the part located at the apex C2 of the fixed substrate 51) of the fixed extraction electrode 563 forms a fixed electrode pad 563P connected to the driver circuit 243.

Note that, in the embodiment, the configuration in which one fixed electrode 561 is provided on the electrode provision surface 511A is shown, however, for example, a configuration in which two electrodes forming concentric circles around the plane center point O are provided (dual electrode configuration) may be employed.

As described above, the reflection film provision part 512 is formed in a nearly cylindrical shape having a diameter dimension smaller than that of the electrode placement groove 511 coaxially with the electrode placement groove 511, and includes the reflection film provision surface 512A of the reflection film provision part 512 opposed to the movable substrate 52.

As shown in FIGS. 5 and 6, the fixed reflection film 54 is provided on the reflection film provision part 512. As the fixed reflection film 54, for example, a metal film of Ag, an alloy film of an Ag alloy, or the like may be used. Further, for example, a dielectric multilayer film with a high-refractive-index layer of TiO₂ and a low-refractive-index layer of SiO₂ may be used. Furthermore, a reflection film formed by stacking a metal film (or an alloy film) on the dielectric multilayer film, a reflection film formed by stacking the dielectric multilayer film on a metal film (or an alloy film), a reflection film formed by stacking a single-layer refractive layer (TiO₂, SiO₂, or the like) and a metal film (or an alloy film), or the like may be used.

In addition, of the surface of the fixed substrate 51 opposed to the movable substrate 52, the surface without the electrode placement groove 511, the reflection film provision part 512, or the electrode extraction groove 511B formed thereon by etching forms a first joining part 513. The first joining part 513 is joined to the movable substrate 52 by the joining film 53.

### Configuration of Movable Substrate

The movable substrate 52 includes the movable part 521 having a circular shape around the plane center point O, a holding part 522 that is coaxial with the movable part 521 and holds the movable part 521, and a substrate outer circumference part 525 provided outside of the holding part 522 in the filter plan view as shown in FIG. 5.

Further, on the movable substrate 52, a cutout part 524 is formed in correspondence with the apex C2 as shown in FIG. 5, and the fixed electrode pad 563P is exposed when the tunable interference filter 5 is seen from the movable substrate 52 side.

The movable part 521 is formed to have a thickness dimension larger than that of the holding part 522, and, for example, formed to have the same dimension as the thickness dimension of the movable substrate 52 in the embodiment. The movable part 521 is formed to have at least a diameter dimension larger than the diameter dimension of the outer circumference edge of the reflection film provision surface 512A in the filter plan view. Further, in the movable part 521, the movable electrode 562 and the movable reflection film. 55 are provided.

The movable electrode 562 is opposed to the fixed electrode 561 via the gap between electrodes G2, and formed in an annular shape, the same shape as that of the fixed electrode 561. Further, a movable extraction electrode 564 extending from the outer circumference edge of the movable electrode 562 toward the apex C1 of the movable substrate 52 is provided on the movable substrate 52. The extending end part (the part located at the apex C1 of the movable substrate 52) of the movable extraction electrode 564 forms the movable electrode pad 564P connected to the driver circuit 243.

The movable reflection film 55 is provided to be opposed to the fixed reflection film 54 via the gap between reflection films G1 in the center part of a movable surface 521A of the movable part 521. As the movable reflection film 55, a reflection film having the same configuration as the above described fixed reflection film 54 is used.

Note that, in the embodiment, as described above, the example in which the amount of gap of the gap between electrodes G2 is larger than the amount of gap of the gap between reflection films G1 is shown, however, not limited to that. For example, depending on the wavelength range of light to be measured such that infrared light or far-infrared light is used as the light to be measured, the amount of gap of the gap between reflection films G1 may be larger than the amount of gap of the gap between electrodes G2.

The holding part 522 is a diaphragm surrounding the movable part 521, and formed to have a thickness dimension smaller than that of the movable part 521. The holding part 522 is more flexible than the movable part 521, and the movable part 521 can be displaced toward the fixed substrate 51 side by slight electrostatic attractive force. In this regard, the movable part 521 has the larger thickness dimension and the larger stiffness than those of the holding part 522, and thereby, even when the holding part 522 is pulled toward the fixed substrate 51 side by the electrostatic attractive force, the shape change of the movable part 521 does not occur. Therefore, the deflection of the movable reflection film 55 provided in the movable part 521 is not produced, and the fixed reflection film 54 and the movable reflection film 55 may be maintained constantly in the parallel condition.

Note that, in the embodiment, the holding part 522 having the diaphragm shape is exemplified, however, not limited to that. For example, a configuration in which beam-like holding parts arranged at equal angle intervals around the plane center point O are provided may be employed.

As described above, the substrate outer circumference part 525 is provided outside of the holding part 522 in the filter plan view. The surface of the substrate outer circumference part 525 opposed to the fixed substrate 51 is opposed to the first joining part 513 and joined to the fixed substrate 51 by the joining film 53.

### Configuration of Imaging Part

The imaging part 242 has a photoelectric conversion element such as a CCD (Charge Coupled Device) element, and outputs the received light as an electric signal to the control circuit part 28 via the driver circuit 243.

### Configuration of Camera Communication Part

The camera communication part 25 communicates with a main body communication part 43 provided in the base unit 4. In the embodiment, the camera communication part 25 is a wireless communication device and communicates with the main body communication part 43 via wireless communication. For the wireless communication, any communication means including, e.g., wireless LAN communication via a relay point such as infrared communication, Bluetooth (registered trademark), and WiFi (registered trademark), or the like may be used.

### Configuration of Direction Detection Sensor

The direction detection sensor 26 is a direction detection unit according to the invention, and includes a combination of a gyro sensor and an acceleration sensor, for example. The direction detection sensor 26 detects the direction of gravitational force by the acceleration sensor while detecting its displacement angle by the gyro sensor, and thereby, detects the tilt angle and the tilt direction with respect to the vertical direction of the spectroscopic camera 2. That is, the sensor detects the imaging direction by the imaging module 24.

### Configuration of Display

As shown in FIGS. 1 and 4, the display 27 is provided at the rear surface side opposite to the imaging window of the casing 21. The display 27 displays e.g., an analysis result of a component analysis or the like as an image under the control of the control circuit part 28.

### Configuration of Control Circuit Part

The control circuit part 28 includes the driver circuit 243 connected to the imaging part 242 and the respective electrode pads 563P, 564P of the tunable interference filter 5, an input circuit that receives input signals from the operation part 23, and various control circuits that control the camera communication part 25, the direction detection sensor 26, the display 27, etc. Further, as shown in FIG. 2, the control circuit part 28 includes a camera control part 281 having a CPU etc. and a memory part 282 having a memory etc.

In the memory part 282, for example, V-λ data representing relationships of wavelengths of light transmitted through the tunable interference filter 5 with the drive voltages applied to the electrostatic actuator 56 of the tunable interference filter 5 is stored. Further, various kinds of programs for controlling the spectroscopic camera are stored in a memory circuit.

Further, the camera control part 281 reads out and executes the various kinds of programs and acquires spectral images. Specifically, the camera control part 281 executes the various kinds of programs, and thereby, functions as a filter control unit 283, an imaging control unit 284, and a display control unit 285.

The filter control unit 283 reads out the voltage corresponding to the wavelength of the spectral image based on the V-λ data stored in the memory part 282, controls the voltage control circuit, and applies the voltage to the electrostatic actuator 56.

The imaging control unit 284 controls the imaging part 242 and acquires spectral images.

The display control unit 285 allows the display 27 to display information of component analysis results or the like transmitted from the main body communication part 43 to the camera communication part 25, for example.

### Configuration of Support Unit

The support unit 3 includes a plurality of support arms 31 (partial support parts). These support arms 31 change the distance between the spectroscopic camera 2 and a mounting part 41 of the base unit 4 and correspond to a distance change unit according to the invention.

Specifically, as shown in FIG. 3, the support unit 3 includes a first arm 31A, a second arm 31B, and a camera attachment part 31C.

One end of the first arm 31A is coupled to the base unit 4 rotatably around a first rotation shaft 311. The other end of the first arm 31A is coupled to the second arm 31B rotatably around a second rotation shaft 312.

One end of the second arm 31B is coupled to the first arm 31A rotatably around the second rotation shaft 312. The other end of the second arm 31B is coupled to the camera attachment part 31C rotatably around a third rotation shaft 313.

One end of the camera attachment part 31C is coupled to the second arm 31B rotatably around the third rotation shaft 313. To the camera attachment part 31C, the attachment part 22 of the spectroscopic camera 2 is detachably attached.

That is, the respective arms 31A, 31B and the camera attachment part 31C change the coupling angles to one another, thereby, change the position of the spectroscopic camera 2, and form a position change part according to the invention.

Further, the support unit 3 includes a position sensor 32 that detects the position of the support unit 3 (a position detection unit according to the invention). Specifically, the position sensor 32 includes a first angle detection sensor 32A that detects the rotation angle with respect to the base unit 4 in the first rotation shaft 311, and a second angle detection sensor 32B that detects the angle of the second arm 31B with respect to the first arm 31A in the second rotation shaft 312.

The angles detected by the position sensor 32 (detection signals in response to the angle) are respectively output to the control part 45 provided in the base unit 4.

Furthermore, a light source part 33 is provided in the second arm 31B. The turning on and off of the light source part 33 are controlled by the control part 45 of the base unit 4.

The light applied from the light source part 33 is light having a near-infrared wavelength (for example, 700 nm to 1500 nm) used for component analysis processing, for example. Further, as the light source part 33, an LED or a laser light source may be used. The LED or the laser light source is used, and thereby, downsizing and power saving of the light source part 33 may be realized.

### Configuration of Base Unit

Next, the configuration of the base unit 4 will be explained.

As shown in FIGS. 1 and 2, the base unit 4 includes the mounting part 41, a mass measurement part 42 (see FIG. 2), the main body communication part 43 (see FIG. 2) as the first communication part according to the invention, a memory part 44 (see FIG. 2), and a control part 45 (see FIG. 2).

The mounting part 41 is a plate-like member having a horizontal mounting surface on which the measuring object is mounted.

Further, a color reference part 411 (see FIG. 1) as a reference part according to the invention is provided on the mounting surface of the mounting part 41. The color reference part 411 includes a plurality of partial reference parts with known reflectances (reference reflectances) for the respective wavelengths (infrared range) necessary in the composition analysis processing. As the plurality of partial reference parts, for example, it is preferable to include high-reflection reference parts with higher reflectances for the respective wavelengths and low-reflection reference parts with lower reflectances for the respective wavelengths. Furthermore, the color reference part 411 having a configuration in which a plurality of partial reference parts with high reflectances for particular wavelengths and low reflectances for other wavelengths are provided and the particular wavelengths of the high reflectances are different in the respective partial reference parts may be employed.

The mass measurement part 42 is provided below the mounting part 41. The mass measurement part 42 measures the mass of the measuring object mounted on the mounting part 41 and outputs the measurement result to the control part 45.

The main body communication part 43 is the same communication device for wireless communication with that of the camera communication part 25 and transmits and receives data between the camera communication part 25 and itself. That is, the part receives the spectral images taken by the spectroscopic camera 2 and the imaging direction of the spectroscopic camera 2, and transmits the component analysis results and the mass measurement results.

Note that, in the embodiment, the example in which the main body communication part 43 is provided in the base unit 4 is shown, however, the part may be provided in the support unit 3.

In the memory part 44, correlation data (for example, calibration curves) representing correlations between feature quantities (absorbance at specific wavelengths) extracted from absorption spectra for the respective components to be analyzed and component content rates is stored. Further, in the memory part 44, various kinds of data used for other component analysis processing (for example, correction values of absorption spectra of the respective components for the temperatures of the measuring object etc.) may be recorded.

The control part 45 includes an operational circuit such as a CPU, and reads various programs stored in the memory part 44 and executes various kinds of processing. Specifically, the control part 45 executes various kinds of processing, and thereby, functions as a light source control unit 451, a spectral image acquisition unit 452, an initial processing unit 453, a distance calculation unit 454 (distance measurement unit), a component analysis unit 455, etc. That is, in the embodiment, the control part 45 functions as a component analysis part.

The light source control unit 451 controls lighting of the light source part 33 provided in the support unit 3.

The spectral image acquisition unit 452 acquires the spectral image from the spectroscopic camera 2 received by the main body communication part 43.

The initial processing unit 453 executes initial processing (calibration) in the component analysis.

The distance calculation unit 454 calculates the distance from the spectroscopic camera 2 to the mounting part 41 based on the detection result by the position sensor 32 provided in the support unit 3, the imaging direction of the spectroscopic camera, etc.

The component analysis unit 455 analyzes the component of the measuring object and calculates the amounts of components and calculates calories based on the acquired spectral image, the measured mass of the measuring object, the calculated distance between the spectroscopic camera 2 and the mounting part 41, etc.

The specific processing of the respective configurations will be described later.

### Operation of Component Analyzing Apparatus

Next, the operation by the above described component analyzing apparatus 1 will be explained as below with reference to the drawings.

When a component analysis is performed by the component analyzing apparatus 1 of the embodiment, first, the respective arms 31A, 31B of the support unit 3 are set to appropriate angles (angles at which the color reference part 411 of the mounting part 41 can be imaged), and an initial processing for calculation of the absorbance is executed.

In the initial processing, the light source part 33 is lighted by the light source control unit 451.

Then, the control part 45 transmits an initial processing start command of execution of initial processing of the spectroscopic camera 2 from the main body communication part 43 to the camera communication part 25. Thereby, the filter control unit 283 of the spectroscopic camera 2 reads out the V-λ data and sequentially changes the voltage applied to the electrostatic actuator 56. For example, the voltage is sequentially changed so that lights in a predetermined near-infrared wavelength range (for example, 700 nm to 1500 nm) may be transmitted through the tunable interference filter 5 at 10 nm intervals. Further, the imaging control unit 284 acquires the spectral images from the imaging part 242 at each time when the voltage is sequentially changed. These spectral images are images of the color reference part 411 imaged with respect to each wavelength. The obtained spectral images are transmitted from the camera communication part 25 to the main body communication part 43.

When receiving the spectral images for the respective wavelengths, the initial processing unit 453 determines the locations of the color reference part 411 in the respective spectral images. The determination of the location of the color reference part 411 may be performed by detecting edge parts in which amount of light largely changes between pixels in the spectral image or the like.

Then, the initial processing unit 453 measures reference amounts of received light I₀ at the respective wavelengths based on the amounts of light in the color reference part 411 of the spectral images of the respective wavelengths, and stores the reference amounts of received light I₀ in the memory part 44.

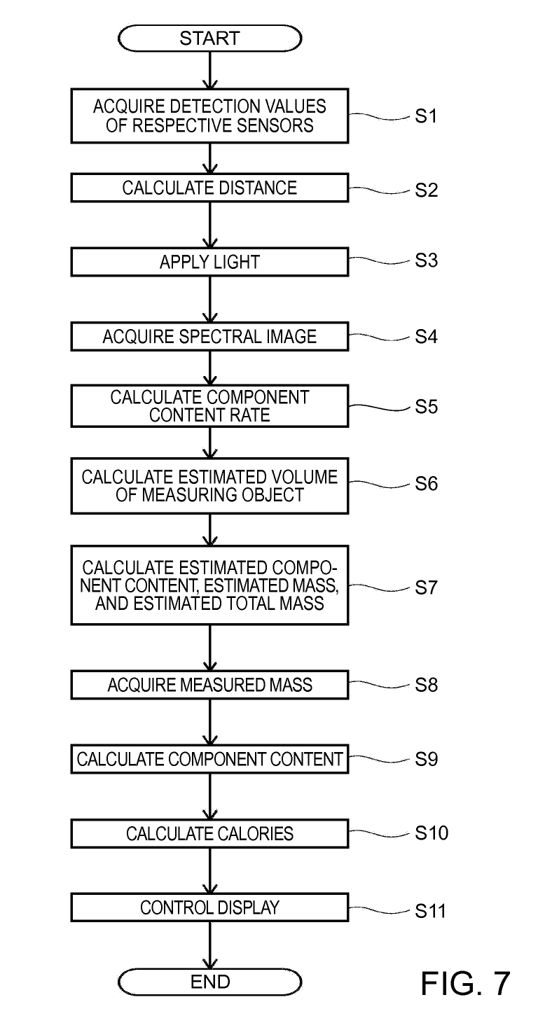

Next, processing when a component analysis of a measuring object is executed after the measuring object is mounted on the mounting part 41 (component analysis processing) will be explained. FIG. 7 is a flowchart showing the component analysis processing.

In the component analysis processing, first, the distance calculation unit 454 calculates the distance from the spectroscopic camera 2 to the mounting part 41.

Specifically, the distance calculation unit 454 acquires detected angles of the position sensor 32 (first angle detection sensor 32A, second angle detection sensor 32B). Further, in the spectroscopic camera 2, the imaging direction of the spectroscopic camera 2 detected by the direction detection sensor 26 is acquired (step S1).

Then, the distance calculation unit 454 calculates the distance from the imaging part 242 of the spectroscopic camera 2 to the mounting part 41 based on the detected angle, the imaging direction detected at step S1 (step S2).

The processing at step S2 will be explained with reference to FIG. 3.

Here, suppose that the angle of the first arm 31A with respect to the base unit 4 detected by the first angle detection sensor 32A is α, the angle of the first arm 31A and the second arm 31B detected by the second angle detection sensor 32B is β, and an angle formed by the imaging direction of the spectroscopic camera 2 and the vertical direction is γ.

Further, the length l₁ from the first rotation shaft 311 to the second rotation shaft 312 in the first arm 31A, the length l₂ from the second rotation shaft 312 to the third rotation shaft 313 in the second arm 31B, and the length l₃ from the third rotation shaft 313 to the attachment location of the spectroscopic camera 2 in the camera attachment part 31C are stored in the memory part 44 in advance. Furthermore, suppose that the distance l₄ from the attachment part 22 to the imaging part 242 in the spectroscopic camera 2 (e.g., the location corresponding to the image center pixel) is registered at the initial use, for example. In addition, suppose that the first rotation shaft 311 is in the same height location as the mounting part 41.

The distance calculation unit 454 respectively calculates the height dimension L₁ (=l₁ sin α) of the second rotation shaft 312 from the mounting part 41, the height dimension L₂ (=l₂ sin(β−α)) from the second rotation shaft 312 to the third rotation shaft 313, and the height dimension L₃ (=(l₃+l₄)sin γ) from the third rotation shaft 313 to the imaging part 242, and calculates the height dimension L′ (=L₁+L₂+L₃) from the mounting part 41 to the imaging part 242.

Then, the distance calculation unit 454 calculates the distance L (L′/cos γ) from the imaging part 242 to the mounting part 41 in the imaging direction.

Then, the light source control unit 451 controls the light source part 33 to apply light to the measuring object (step S3). Further, the spectral image acquisition unit 452 transmits a request signal for requesting acquisition of the spectral image to the spectroscopic camera 2.

When receiving the request signal, the filter control unit 283 of the spectroscopic camera 2 refers to the V-λ data stored in the memory part 282, sequentially reads out the voltages corresponding to the target wavelengths, and applies the voltages to the electrostatic actuator 56. Further, the imaging control unit 284 acquires the spectral image from the imaging part 242 at each time when the voltage is sequentially changed (step S4). The taken spectral image is transmitted from the camera communication part 25 to the main body communication part 43 and stored in the memory part 44.

Note that the target wavelength of the acquired spectral image may be set according to the component for which the analysis processing is performed by the component analyzing apparatus 1, or appropriately set by a person who makes measurement. For example, when amounts of components of fat, sugar, protein, and water and calories of a food are detected by the component analyzing apparatus, it is only necessary that a wavelength at which the feature quantities for at least fat, sugar, protein, and water can be obtained is set as the target wavelength, and the spectral image of the target wavelength is acquired at step S4. Further, the spectral images at predetermined wavelength intervals (e.g., 10 nm intervals) may be sequentially acquired.

Further, when a temperature sensor for measuring the temperature of the measuring object is provided in the component analyzing apparatus 1, the target wavelength of the acquired spectral image may be set based on the temperature. In this case, the temperatures at the respective points (the respective pixels of the taken image) may be detected from the temperature distribution of the measuring object, and the respective measuring object wavelengths may be corrected based on the detected temperatures.

For example, at reference temperature T₀, when the absorbance of the wavelength λ_{A0} changes depending on the content rate of component A, the feature quantity of the component A at the reference temperature T₀ is the absorbance of the wavelength λ_{A0}. However, at temperature T₁, the absorbance of the wavelength λ₁ may change depending on the content rate of the component A and, in this case, the feature quantity of the component A at the temperature T₁ is the absorbance of the wavelength λ₁. Particularly, regarding water, it is known that the absorption spectrum largely changes due to the temperature change, and it is preferable to correct the wavelength at which the feature quantity is detected for analyses of the respective components.

In the configuration in which the temperature sensor is provided as described above, the correction values of the respective components for the respective temperatures are stored in the memory part 44 in advance. Then, the correction value is read out and the wavelength λ_{A0} is multiplied by the correction value, and the target wavelength λ₁ at which the feature quantity for the temperature T₁ is detected is calculated. Further, in the case where the temperatures are different depending on the parts of a test object, the target wavelengths are respectively calculated in correspondence with the temperatures of the respective parts.

Then, the component analysis is performed by the component analysis unit 455.

Here, in the embodiment, the case where a plurality of measuring objects are mounted on the mounting part 41 is exemplified. That is, in the embodiment, by using the mass measured by the mass measurement part 42 and the estimated mass estimated based on the distance calculated by the distance calculation unit 454, the component analysis of the plurality of measuring objects may be performed without individual mounting of the plurality of measuring objects on the mounting part 41 or performance of a plurality of measurements.

Specifically, the component analysis unit 455 specifies the pixel range in which the respective measuring objects are projected and calculates the content rates of the respective components in the respective measuring objects (step S5). The specification of the measuring object food may be specified based on the imaged spectral images. For example, as a method of specifying the measuring object, the image processing technology in related art may be used, and the pixel range in which the measuring object is projected is specified by edge detection within the image or the like. Note that the method of specifying the measuring object is not limited to that. For example, when the shape feature values of the measuring objects are stored in the memory part 44, the measuring object may be specified by analyzing the image based on the shape feature value.

Then, the component analysis unit 455 calculates average values of the content rates in the respective pixels of the specified respective measuring objects with respect to the respective components, and uses them as component content ratios (mg/cm³) in the respective measuring objects. Note that a plurality of pixels may be picked up from the pixel range of the specified measuring objects, and the component content rates analyzed with respect to the pixels may be averaged.

As a method of obtaining the content rate, for example, absorbance A_{λ} of the wavelength λ in each pixel is calculated using the following formula (1) based on the reference amount of received light I₀ and the amount of received light I_{λ} in each pixel of the imaged spectral image of the wavelength λ.

A_{λ}=−log(I_{λ}/I₀) (1)

Then, the component analysis unit 455 analyzes the content rates of the respective components based on the calculated absorbance A_{λ} and the correlation data stored in the memory part 44. As a method of analyzing the component content rates, the chemometrics method used in related art may be employed. As the chemometrics method, for example, multiple regression analysis, principal component regression analysis, partial least square method, or the like may be used. Note that the respective analysis methods using the chemometrics method are known technologies, and the explanation here is omitted.

Then, the component analysis unit 455 calculates sizes (estimated volumes) of the respective measuring objects from the distance L from the imaging part 242 to the mounting part 41 calculated at step S2 and the pixel ranges of the respective measuring objects specified in the respective spectral images (step S6).

Then, the component analysis unit 455 calculates the respective component contents (g) of the respective measuring objects by products of the estimated volumes (cm³) of the respective measuring objects calculated at step S6 and the respective component content rates (mg/cm³) of the respective measuring objects calculated at step S5, and calculates estimated masses (g) of the respective measuring objects and an estimated total mass (g) as the sum of the estimated masses of the respective measuring objects from the sum of the respective component contents with respect to the respective measuring objects (step S7).

Then, the component analysis unit 455 acquires the measured masses (g) of the measuring objects measured by the mass measurement part 42 (step S8).

Then, the component analysis unit 455 calculates correction values (measured masses/estimated masses) from the measured masses acquired at step S8 and the estimated masses of the respective measuring objects calculated at step S8, and multiplies the respective component contents (g) of the respective measuring objects calculated at step S8 by the correction values (step S9).

In the above described manner, when the plurality of measuring objects are mounted on the mounting part 41, the component contents contained in these measuring objects may be easily calculated.

The following table 1 shows examples of values calculated by the processing from step S5 to step S9 when measuring objects A, B are mounted on the mounting part 41.

TABLE 1

| | | | Measuring | Measuring | |
|---|---|---|---|---|---|
| Step | Analysis contents | | object A | object B | Unit |
| S5 | Component | Water | 1190 | 95 | mg/cm³ |
| | analysis | Carbohydrate | 300 | 117 | mg/cm³ |
| | result by | Protein | 23 | 23 | mg/cm³ |
| | near-infrared | Fat | 1.5 | 11 | mg/cm³ |
| | light (a) | | | | |
| S6 | Volume estimated from image | | 65.4 | 240 | cm³ |
| | (b) | | | | |
| S7 | Estimated | Water | 77.8 | 22.8 | g |
| | component | Carbohydrate | 19.6 | 28.1 | g |
| | content | Protein | 1.5 | 5.5 | g |
| | calculation | Fat | 0.1 | 2.6 | g |
| | (c) = (a) × (b) | | | | |
| S7 | Estimated mass (d) | | 99.0 | 59.0 | g |
| S7 | Estimated total mass | | 158.1 | | g |
| | Total of (e) = (d) | | | | |
| S8 | Measured value of mass meter | | 180 | | g |
| | (f) | | | | |
| S9 | Corrected | Water | 88.6 | 26.0 | g |
| | Component | Carbohydrate | 22.3 | 32.0 | g |
| | contents | Protein | 1.7 | 6.3 | g |
| | (g) = (c) × | Fat | 0.1 | 3.0 | g |
| | (f)/(e) | | | | |

Further, the component analysis unit 455 calculates calories of the measuring objects based on the expression (2) from the calculated weights of the respective components (weights of fat, sugar, protein) (step S10).

calorie (kcal)=fat mass (g)×9+protein mass (g)×4+sugar mass (g)×4 (2)

Then, the component analysis unit 455 transmits the contents of the respective components calculated at step S9 and the calories of the measuring objects calculated at step S10 to the spectroscopic camera 2 for display on the display 27 of the spectroscopic camera 2 (step S11).

### Advantages of First Embodiment

In the embodiment, the mass measurement part 42 is provided in the base unit 4, and thereby, the correct mass of the measuring object may be measured.

Further, the spectroscopic camera 2 is supported by the support unit 3 provided on the base unit 4, and thereby, there is no blur of the spectral image due to camera shake or the like, the distance from the spectroscopic camera 2 to the mounting part 41 of the base unit 4 is maintained constant, and the spectral image of the measuring object may be acquired at the distance. Therefore, the correct size of the measuring object may be calculated from the distance from the spectroscopic camera 2 to the mounting part 41, and the component contents of the measuring object may be calculated with high accuracy based on the mass of the measuring object and the amounts of light of the respective pixels of the spectral image.

Furthermore, the component analysis may be performed by the simple configuration in which the spectroscopic camera 2 is supported by the support unit 3 with respect to the base unit 4, and simplification of the configuration may be realized compared to an apparatus in related art in which a mounting part is provided within a casing having a large chamber for component analysis.

In the embodiment, the support unit 3 includes the first arm 31A, the second arm 31B, and the camera attachment part 31C that can change the coupling angles to one another. Accordingly, the coupling angles of the respective arms 31A, 31B and the camera attachment part 31C are adjusted, and thereby, the distance from the mounting part 41 to the spectroscopic camera 2 and the imaging direction may be freely changed so that the measuring object may be within the spectral image according to the size of the measuring object.

Further, in the embodiment, the position sensor 32 that detects the rotation angles of the respective rotation shafts 311, 312, 313 is provided, and the control part 45 includes the distance calculation unit 454 for calculating the distance from the spectroscopic camera 2 to the mounting part 41 based on the detected angle by the position sensor 32 and the imaging direction detected by the direction detection sensor provided in the spectroscopic camera 2.

Accordingly, as described above, even when the position of the support unit 3 is changed, the correct distance from the spectroscopic camera 2 to the mounting part 41 may be calculated, and the size of the measuring object may be calculated.

In the embodiment, the spectroscopic camera 2 is provided detachably with respect to the camera attachment part 31C. Accordingly, the spectroscopic camera 2 is detached, and thereby, the maintenance of the spectroscopic camera 2 may be preferably performed. Further, expansion of use including attachment of the existing spectroscopic camera 2 to the camera attachment part 31C may be realized.

In the embodiment, the main body communication part 43 is provided in the base unit 4 and the camera communication part 25 is provided in the spectroscopic camera 2. Accordingly, as described above, even when the spectroscopic camera 2 has the detachable configuration, the spectral image taken in the spectroscopic camera 2 may be output to the control part 45 of the base unit 4 without other wiring.

In the embodiment, the color reference part 411 is provided in part of the mounting part 41. Accordingly, at calibration, it is not necessary to prepare another reference object having known reflectance, and appropriate calibration may be performed by the color reference part 411 on the mounting part 41.

In the embodiment, in the spectroscopic camera 2, the Fabry-Perot etalon element is used. Accordingly, downsizing of the spectroscopic camera 2 may be realized without using a larger spectroscopic unit such as an AOTF or an LCTF, for example.

The light source part 33 is provided in the second arm 31B. Generally, when the light applied from the light source part 33 is regularly reflected by the measuring object and imaged, the amount of light is abnormal in the part of the regular reflection and correct component analysis is impossible. On the other hand, in the embodiment, the position of the second arm 31B is changed and the light application direction from the light source part 33 is also changed, and thereby, the location of the regular reflection part in the measuring object is also changed. Therefore, the position of the support unit 3 may be changed so that the regular reflection part may not exist, and the high-accuracy component analysis may be performed.

In the embodiment, even when a plurality of measuring objects are mounted on the mounting part 41, the estimated amounts of component contents of the respective measuring objects are corrected to the component contents based on the actual mass measurement values by the processing at step S5 to step S9, and thereby, the component measurement results with higher accuracy may be obtained. Therefore, the more accurate values may be calculated in the calorie calculation etc.

### Second Embodiment

In the above described first embodiment, the component analysis of the measuring object is performed with the distance from the spectroscopic camera 2 to the base unit 4 kept constant.

On the other hand, there are cases where the light of the light source part 33 is regularly reflected by the measuring object and it is impossible to appropriately perform the component analysis for the part of the regular reflection. In the first embodiment, the location of the regular reflection part in the measuring object may be moved not to enter the measuring object by changing the position of the support unit 3. However, for example, the regular reflection part may exist somewhere within depending on the size of the measuring object or the like. In this case, the accuracy of the component analysis with respect to the regular reflection part may be lowered. In the second embodiment, to cope with the case, the amount of light of the regular reflection part is corrected to an appropriate value, and thereby, the more accurate component analysis is performed.

That is, in the embodiment, the position of the support unit 3 is changed, and thereby, the light output direction of the light source part 33 provided in the support unit 3 is changed and two spectral images having different regular reflection parts for the respective wavelengths are acquired. In this regard, the distance from the spectroscopic camera 2 to the base unit 4 is correctly calculated, and thereby, even when the position of the support unit 3 is changed, the locations to which the respective pixels of the acquired two spectral images correspond in the measuring object may be easily determined.

Note that, for the following explanation of the embodiments, the same configuration and processing with those of the first embodiment have the same signs and their explanation will be omitted or simplified.

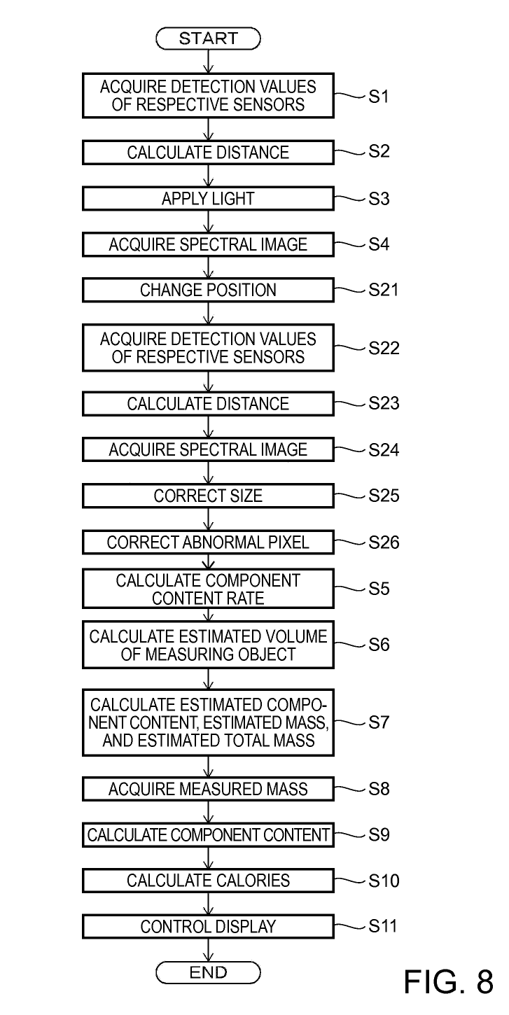

FIG. 8 is a flowchart showing component analysis processing in the embodiment.

In the embodiment, as shown in FIG. 8, the spectral images for the respective wavelengths (hereinafter, may be referred to as “first spectral images”) are acquired at step S4, and then, the position of the support unit 3 is changed (step S21).

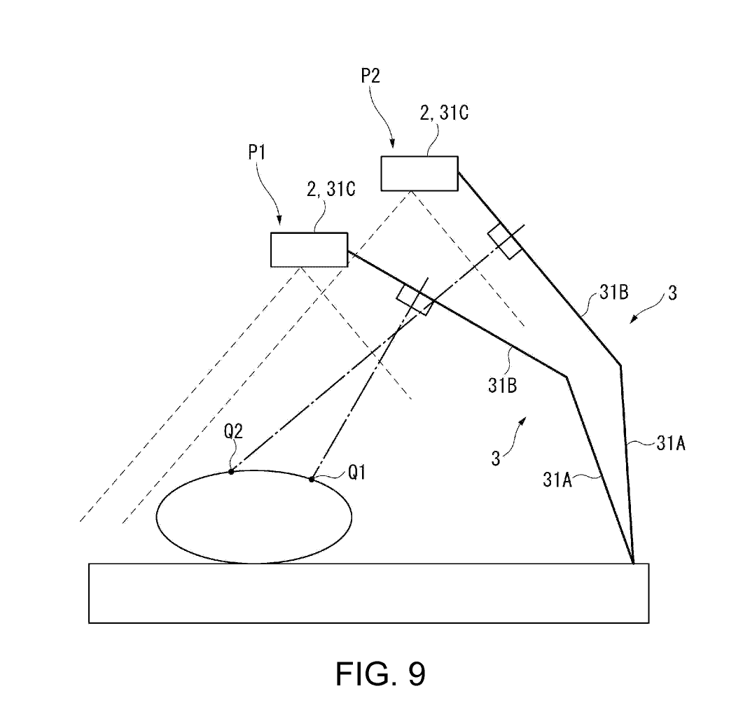

FIG. 9 is a schematic diagram showing position change of the support unit 3.

As shown in FIG. 9, when the position of the support unit 3 is changed, the output direction of the light output from the light source part 33 changes. For example, as shown in FIG. 9, supposing that the position of the support unit 3 when the spectral images are acquired at step S4 is P1, when the position of the support unit 3 is changed to P2 at step S21, the regular reflection part in the measuring object moves from Q1 to Q2.

Then, the distance calculation unit 454 acquires the detected angles of the position sensor 32 (first angle detection sensor 32A, second angle detection sensor 32B) like steps S1, S2 (step S22), and calculates the distance from the imaging part 242 of the spectroscopic camera 2 to the mounting part 41 based on the detected angles and imaging direction (step S23).

Then, the spectral image acquisition unit 452 transmits a request signal for requesting acquisition of spectral images to the spectroscopic camera 2. Thereby, the filter control unit 283 of the spectroscopic camera 2 performs the same processing as that at step S4 and spectral images for the respective wavelengths (hereinafter, may be referred to as “second spectral images”) are acquired (step S24).

Then, the component analysis unit 455 performs image correction on the acquired second spectral images based on the distance calculated at step S2 (first distance) and the distance calculated at step S23 (second distance). That is, the component analysis unit 455 calculates a distance ratio between the first distance and the second distance (first distance/second distance), magnifies or reduces the second spectral images in response to the distance ratio to make the sizes of the measuring object of the first spectral images and the measuring object of the second spectral images to be equal, and then, performs trimming processing (step S25).

Then, the component analysis unit 455 detects pixels (abnormal pixels) corresponding to the regular reflection part Q1 of the first spectral images and corrects the amounts of light of the abnormal pixels (step S26).

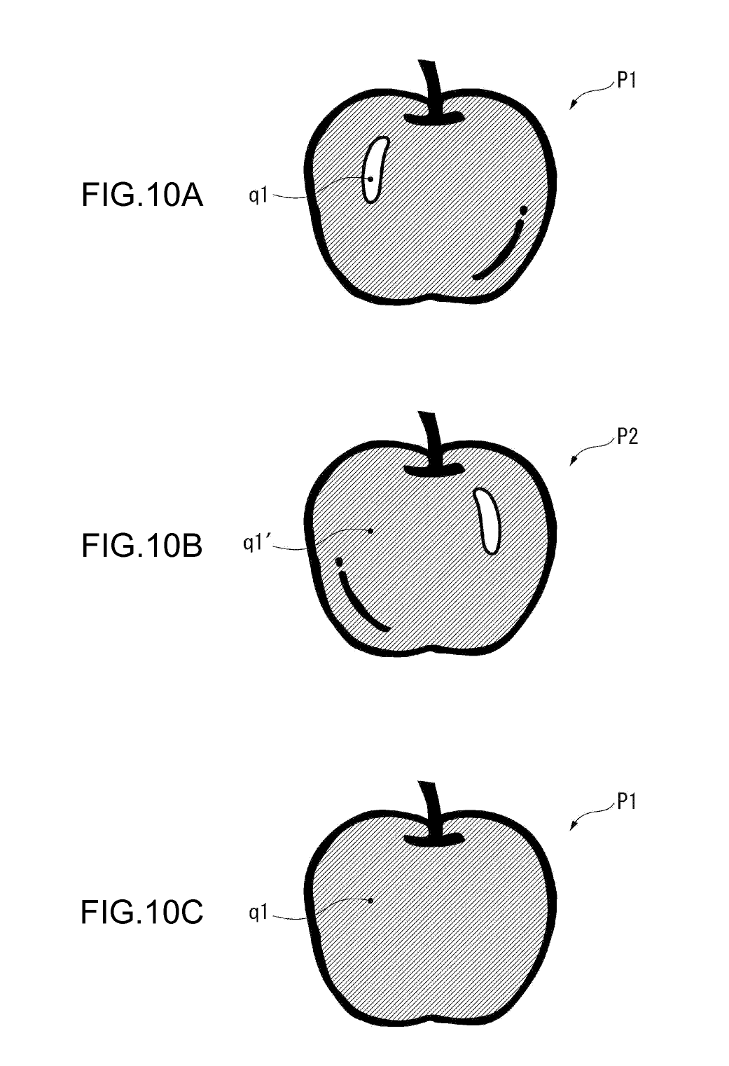

FIGS. 10A to 10C show examples of the first spectral image, the second spectral image after size correction, and the corrected spectral image. FIG. 10A shows an example of the first spectral image taken in the position P1, FIG. 10B shows an example of the second spectral image taken in the position P2, and FIG. 10C shows an example of the corrected first spectral image.

Specifically, the component analysis unit 455 calculates ratios of the amounts of light of the respective pixels of the first spectral images for the respective wavelengths to the reference amounts of received light I₀ (reflectance ratios). Then, the component analysis unit 455 detects the pixels having the reflectance ratios larger than “1”. Note that, if there is no pixel having the reflectance ratio larger than “1”, the pixel correction is not performed, but the processing at step S5 may be performed.

When detecting an abnormal pixel (for example, the pixel q1 in FIG. 10A), the component analysis unit 455 stores the pixel location of the abnormal pixel in the memory part 44.

On the other hand, when detecting the abnormal pixel in the first spectral image, the component analysis unit 455 acquires amount of light of the pixel (pixel q1′ in FIG. 10B) of the second spectral image corresponding to the pixel location. Then, the component analysis unit 455 replaces the amount of light of the abnormal pixel q1 of the first spectral image by the amount of light of the corresponding pixel q1′ of the second spectral image as shown in FIG. 10C.

Then, the processing from step S5 to S11 is performed in the same manner as that of the first embodiment based on the first spectral images having the corrected amounts of light.

Further, in the embodiment, a configuration of automatically changing the position of the support unit 3 may be provided in addition to the configuration of the first embodiment.

For example, gears rotating when a drive force from a drive motor (not shown) is transmitted are provided on the respective rotation shafts 311, 312, 313 of the support unit 3, driving of the drive motor is controlled by the control of the control part 45, and thereby, the rotation angles of the arms 31A, 31B and the camera attachment part 31C on the respective rotation shafts are changed.

In this case, it is preferable that the control part 45 changes the position of the support unit 3 at the step S21 so that the imaging direction detected by the direction detection sensor 26 of the spectroscopic camera 2 may be the same direction as that when the spectral images are acquired at step S11. In this case, pixel shift due to changes in imaging direction may be suppressed, the regular reflection part in the first spectral image and the pixel location of the second spectral image corresponding to the regular reflection part may be detected more accurately, and the highly accurate component analysis may be performed.

### Advantages of Second Embodiment

In the embodiment, the light source part 33 is provided in the second arm 31B of the support unit 3 like the first embodiment.

Therefore, even when the abnormal pixel corresponding to the regular reflection part in which the light from the light source part 33 is regularly reflected exists in the first spectral image, the location of the regular reflection part of the measuring object may be changed by changing the position of the support unit 3. Therefore, the second spectral image after position change is acquired and the abnormal pixel in the first spectral image is replaced by the amount of light of the pixel of the second spectral image corresponding to the abnormal pixel, and thereby, the component analysis with high accuracy may be performed on the regular reflection part.

### Third Embodiment

Next, the third embodiment according to the invention will be explained with reference to the drawings.

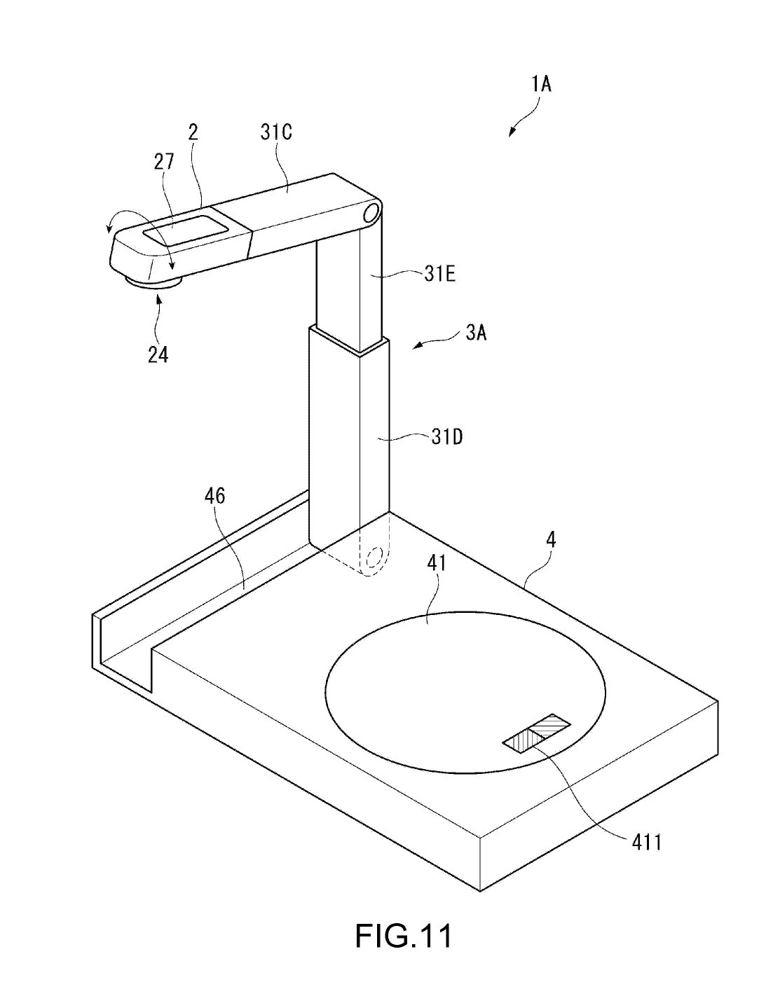

FIG. 11 is a perspective view showing a schematic configuration of a component analyzing apparatus of the embodiment.

As shown in FIG. 11, in a component analyzing apparatus 1A in the embodiment, a housing part 46 that houses a support unit 3A and the spectroscopic camera 2 is provided in the base unit 4.

The support unit 3A includes a first support 31D rotatably and axially supported inside of the housing part 46, a second support 31E extending and retracting with respect to the first support 31D, and the camera attachment part 31C rotatably and axially supported with respect to the second support 31E.

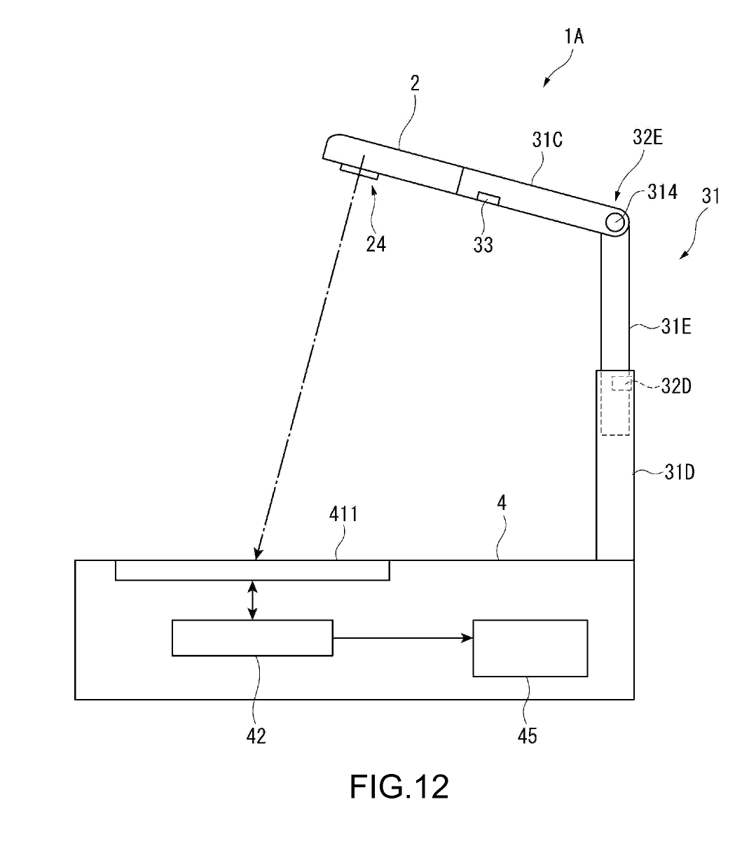

FIG. 12 schematically shows the support unit of the embodiment.

As shown in FIG. 12, the first support 31D is formed to have a hollow shape and is movable along the axial direction of the first support 31D when the second support 31E is inserted into the first support 31D. Further, a movement detection sensor 32D that detects the movement of the second support 31E is provided within the first support 31D. Further, a rotation angle detection sensor 32E is provided in a shaft part 314 that axially supports the camera attachment part 31C in the second support 31E. These movement detection sensor 32D and rotation angle detection sensor 32E form a position detection unit according to the invention.

Furthermore, it is preferable that the spectroscopic camera 2 is rotatably provided with respect to the camera attachment part 31C around a rotation shaft in the longitudinal direction of the camera attachment part 31C. Thereby, as shown in FIG. 11, the spectroscopic camera 2 is rotated in the direction toward the mounting part 41 so that the spectral images of the measuring object may be taken.

In the embodiment, the second support 31E is retracted into the first support 31D, the camera attachment part 31C is rotated toward the second support 31E side, the first support 31D is rotated toward the base unit 4 side, and thereby, the support unit 3A may be housed within the housing part 46.

Therefore, the component analyzing apparatus 1A may be made smaller and its portability may be improved.

### Other Embodiments

Note that the invention is not limited to the above described embodiments, but the invention includes alterations, improvements, etc. within the range in which the purpose of the invention can be achieved.

For example, in the first embodiment, the configuration in which the arms 31A, 31B as the distance change unit are provided in the support unit 3 as the distance change unit, and the distance from the spectroscopic camera 2 to the mounting part 41 can be changed by the arms 31A, 31B has been exemplified. Further, in the second embodiment, the configuration in which the first support 31D, the second support 31E, and the camera attachment part 31C are provided as the distance change unit and the distance from the spectroscopic camera 2 to the mounting part 41 can be changed by the arms 31A, 31B has been exemplified.

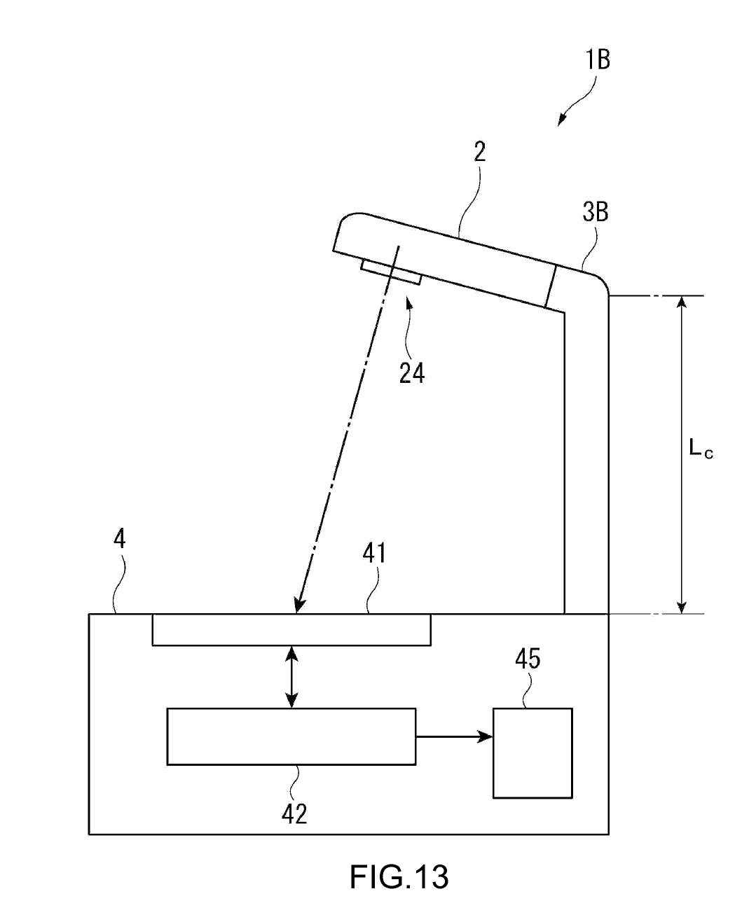

In contrast, a configuration as shown in FIG. 13 may be employed. FIG. 13 is a schematic diagram showing an example of a component analyzing apparatus of a modified example in the invention.

That is, in the example shown in FIG. 13, in a component analyzing apparatus 1B, the distance change unit is not provided in a support unit 3B and the location to which the spectroscopic camera 2 is attached is fixed. Therefore, in the support unit 3B, the distance from the base unit 4 to the location to which the spectroscopic camera 2 is attached is a fixed value Lc and the position sensor is unnecessary.

In this case, the distance calculation unit 454 in the control part 45 may easily calculate the distance from the spectroscopic camera 2 to the mounting part 41 using a trig function based on the imaging direction detected by the spectroscopic camera 2 because the attachment location of the spectroscopic camera 2 is the fixed location. Further, the spectroscopic camera 2 may be fixed to the support unit 3B and, in this case, the imaging direction is also fixed. Therefore, the distance from the spectroscopic camera 2 to the mounting part 41 is measured in advance, and thereby, other calculation of the distance is unnecessary and simplification of the processing may be realized.

Furthermore, in the first embodiment, the distance from the spectroscopic camera 2 to the mounting part 41 is calculated by the distance calculation unit 454 using the angles of the respective arms 31A, 31B detected by the position sensor 32, however, the calculation is not limited to that.

For example, in the first embodiment, a configuration in which a plurality of locking portions are provided on the first arm 31A, an engagement portion that engages with the locking portion is provided on the second arm 31B, and angles of the second arm 31B with respect to the first arm 31A when the engagement portion is engaged with the locking portions are stored in the memory part 44 may be employed. In this case, as the position sensor, a switching device that detects the location of the locking portion with which the engagement portion is locked or the like may be used. In addition, the distance calculation unit 454 reads out the angle corresponding to the locking location of the engagement portion detected by the switching device from the memory part 44 and calculates the distance from the spectroscopic camera 2 to the mounting part 41 using the angle.

Note that the same applies to the first arm 31A with respect to the base unit 4, and a configuration that can be rotated to the angle preset by the engagement portion and the locking portion may be employed. The same applies to the second and the third embodiments.

In the first embodiment, the distance from the spectroscopic camera 2 to the mounting part 41 is calculated using the position sensor 32 and the direction detection sensor 26, however, the calculation is not limited to that. For example, a position sensor that detects the angle of the camera attachment part 31C with respect to the second arm 31B may be provided and the distance calculation unit 454 may calculate the distance from the spectroscopic camera 2 to the mounting part 41 based on the detected angles from the three position sensors.

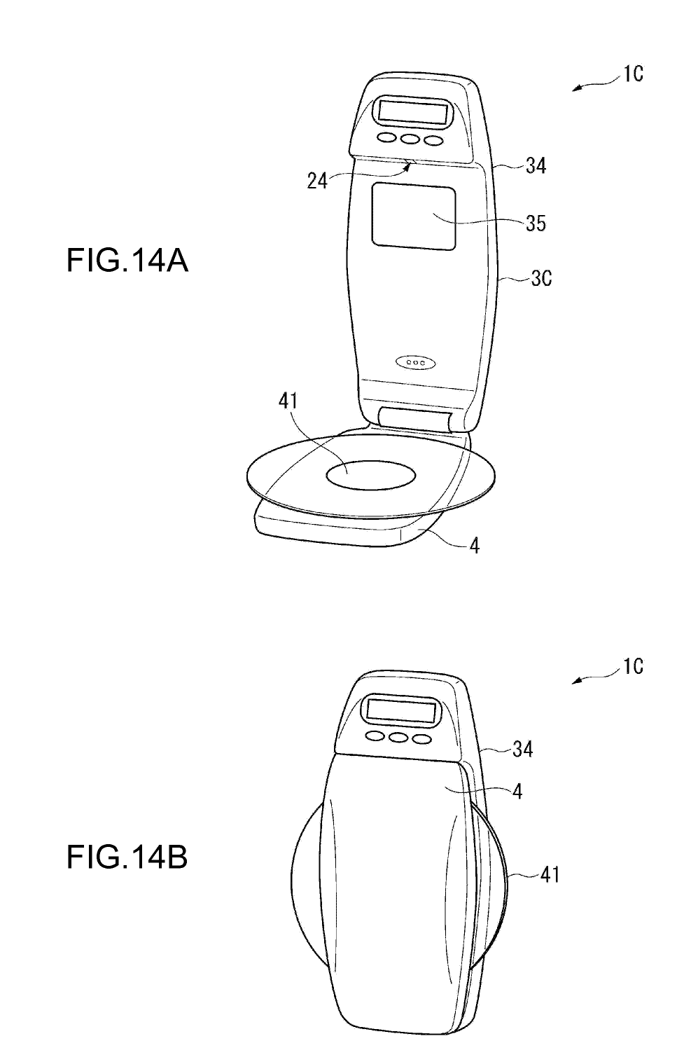

Further, in the embodiments, the arms and the supports have been exemplified as the support units, however, they are not limited to those. For example, a configuration as shown in FIGS. 14A and 14B may be employed.

In a component analyzing apparatus 1C shown in FIG. 14A, a support unit 3C is provided rotatably with respect to the base unit 4. The support unit 3C has a casing 34 and the spectroscopic camera 2 is incorporated in the casing 34. Further, as shown in FIGS. 14A and 14B, a display 35 is provided in a part of the casing 34.

In the configuration, the imaging direction of the spectroscopic camera 2 is fixed with respect to the support unit 3C, and the distance calculation unit may calculate the distance from the spectroscopic camera 2 to the mounting part 41 from the rotation angle of the support unit 3C.

Furthermore, as shown in FIG. 14B, the support unit 3C is rotated toward the mounting part 41 side, and thereby, the apparatus may be made even smaller and the portability may be further improved because no arm or support is provided.

In addition, in the component analyzing apparatus 1C in which the spectroscopic camera 2 is incorporated, various kinds of data (e.g., V-λ data etc.) for driving the spectroscopic camera 2 may be stored in the memory part at the base unit 4 side and the control of the spectroscopic camera 2 may be performed by the control part provided at the base unit 4 side.

In the above described respective embodiments, the configuration in which the main body communication part 43 and the camera communication part 25 communicate with each other via wireless communication has been exemplified, however, the communication is not limited to that. For example, a configuration in which, when the spectroscopic camera 2 is attached to the support unit 3 and connected to a terminal provided in the support unit 3, and thereby, the main body communication part and the camera communication part are connected to enable communication with each other may be employed.

In the above described respective embodiments, the tunable interference filter 5 has the electrostatic actuator 56 that varies the gap dimension between the reflection films 54, 55 by voltage application, however, the filter is not limited to that.

For example, an electrostatic actuator in which a first dielectric coil is provided in place of the fixed electrode 561 and a second dielectric coil or a permanent magnet is provided in place of the movable electrode 562 may be used.

Further, a configuration using a piezoelectric actuator in place of the electrostatic actuator 56 may be employed. In this case, for example, a lower electrode layer, a piezoelectric film, and an upper electrode layer are stacked in the holding part 522 and a voltage applied between the lower electrode layer and the upper electrode layer is varied as an input value, the piezoelectric film is expanded and contracted, and thereby, the holding part 522 may be bent.

Furthermore, in the embodiments, as the Fabry-Perot etalon, the tunable interference filter 5 in which the fixed substrate 51 and the movable substrate 52 are joined to be opposed to each other, the fixed reflection film 54 is provided on the fixed substrate 51, and the movable reflection film 55 is provided on the movable substrate 52 has been exemplified, however, the filter is not limited to that.

For example, a configuration in which the fixed substrate 51 and the movable substrate 52 are not joined and a gap change part that changes the gap between reflection films of the piezoelectric element or the like is provided between the substrates may be employed.

Further, the configuration is not limited to the example having the two substrates. For example, a tunable interference filter in which two reflection films are stacked on one substrate via a sacrifice layer and a gap is formed by removing the sacrifice layer by etching or the like may be used.

Furthermore, as the spectroscopic device, for example, an AOTF or an LCTF may be used. Note that, in this case, it may be difficult to downsize the spectroscopic camera 2, and the Fabry-Perot etalon is preferably used.

The other specific structures when the invention is embodied may be appropriately changed to other structures etc. within the range in which the purpose of the invention can be achieved.

The entire disclosure of Japanese Patent Application No. 2013-239661, filed Nov. 20, 2013 is expressly incorporated by reference herein.

What is claimed is:

1. A component analyzing apparatus comprising:
a mounting part on which a measuring object is capable of mounting;
a mass measurement part that measures a mass of the measuring object;
a spectroscopic imaging unit that takes a spectral image of the measuring object; and
a component analysis part that analyzes components of the measuring object based on the spectral image and the mass.

2. The component analyzing apparatus according to claim 1, further comprising:
a base unit including the mounting part and the mass measurement part; and
a support unit that supports the spectroscopic imaging unit in a location such that a direction in which the spectral image is taken in the spectroscopic imaging unit is a direction toward the mounting part and a distance between the mounting part and the spectroscopic imaging unit is a predetermined distance.

3. The component analyzing apparatus according to claim 2, further comprising:
a distance change unit that changes the distance between the mounting part and the spectroscopic imaging unit; and
a distance measurement unit that measures the distance between the mounting part and the spectroscopic imaging unit.

4. The component analyzing apparatus according to claim 3, wherein the distance change unit includes a position change part that changes a position of the support unit with respect to the base unit.

5. The component analyzing apparatus according to claim 4, wherein the distance change unit includes a position detection unit that detects the position of the support unit with respect to the base unit, and
the distance measurement unit calculates the distance between the spectroscopic imaging unit and the mounting part based on the detected position.

6. The component analyzing apparatus according to claim 4, further comprising a direction detection unit that detects the direction in which the spectral image is taken,
wherein the distance measurement unit calculates the distance between the spectroscopic imaging unit and the mounting part based on the detected direction in which the spectral image is taken.

7. The component analyzing apparatus according to claim 4, wherein the support unit includes a plurality of partial support parts rotatably coupled to each other.

8. The component analyzing apparatus according to claim 4, wherein the support unit is rotatably provided with respect to the base unit, and
the base unit includes a housing part in which the support unit can be folded and housed when the support unit is rotated toward the base unit side.

9. The component analyzing apparatus according to claim 4, further comprising a light source part provided in a part of the support unit and applying light to the measuring object.

10. The component analyzing apparatus according to claim 2, wherein the spectroscopic imaging unit is detachable from the support unit.

11. The component analyzing apparatus according to claim 10, wherein the component analysis part is provided in the base unit or the support unit, and has a first communication part that makes wireless communication with the spectroscopic imaging unit, and
the spectroscopic imaging unit has a second communication part that makes wireless communication with the first communication part.

12. The component analyzing apparatus according to claim 1, wherein the mounting part includes a reference part having reference reflectance.

13. The component analyzing apparatus according to claim 1, wherein the spectroscopic imaging unit includes a tunable Fabry-Perot etalon that light from the measuring object enters, selects light having a predetermined wavelength from the incident light, and can change the predetermined wavelength, and a light receiving element that receives the light output from the Fabry-Perot etalon.

14. The component analyzing apparatus according to claim 1, further comprising a memory unit in which correlation data between a feature quantity extracted from an absorption spectrum of a component to be analyzed and a component content rate of the component to be analyzed is stored,
wherein, when a plurality of the measuring objects are mounted on the mounting part, the component analysis part calculates the content rates of the component to be analyzed contained in the respective plurality of the measuring objects calculated based on amounts of light of the respective pixels of the spectroscopic imaging unit and the correlation data, calculates estimated volumes of the respective plurality of the measuring objects calculated based on the distance between the mounting part and the spectroscopic imaging unit, estimated weights of the component to be analyzed contained in the respective plurality of the measuring objects based on the content rates of the component to be analyzed contained in the respective plurality of the measuring objects, and estimated weights of the respective plurality of the measuring objects, and corrects the estimated weights of the component to be analyzed contained in the respective plurality of the measuring objects based on a sum of the estimated weights of the respective plurality of the measuring objects and a ratio to the mass measured by the mass measurement part.
